(19) United States
(12) Patent Application Publication
(10) Pub. No.: US 2024/0423704 A1
(43) Pub. Date: Dec. 26, 2024

(54) ETHMOIDAL NERVE TREATMENT SYSTEM AND METHOD

(71) Applicant: Aerin Medical Inc., Sunnyvale, CA (US)

(72) Inventor: Scott J. Wolf, Menlo Park, CA (US)

(21) Appl. No.: 18/737,401

(22) Filed: Jun. 7, 2024

Related U.S. Application Data
(60) Provisional application No. 63/510,354, filed on Jun. 26, 2023.

(51) Int. Cl. A61B 18/14; A61B 18/00; A61B 18/02

(52) U.S. Cl. CPC A61B 18/1485; A61B 18/02; A61B 2018/00023; A61B 2018/0022; A61B 2018/00327; A61B 2018/00434; A61B 2018/00577

(57) ABSTRACT

A method for treating a nasal airway to ameliorate one or more symptoms of rhinitis in a patient may involve advancing a treatment tip of a treatment device into a nostril of the patient, contacting nasal mucosa with the treatment tip at a first location overlying an anterior ethmoidal nerve, delivering energy from the treatment tip, to change a property of the anterior ethmoidal nerve, and removing the treatment tip from the nostril.

### FIELD

The present application is related to medical devices and methods. More specifically, the application is related to a system and method for treating nasal airway nerve tissue, including but not limited to the anterior ethmoidal nerve.

### BACKGROUND

Medical devices that use energy to treat tissue in the body are used for a wide variety of procedures, to treat many different conditions and injuries. The assignee of the present application, for example, has developed a number of devices, systems and methods for treating airway tissue, specifically nasal airway tissue in some embodiments, by delivering energy to the tissue. For example, in some embodiments, an energy delivery device is inserted into a patient's nostril to deliver energy (and sometimes mechanical force) to tissue in the nose. This type of energy delivery treatment, for example radiofrequency (RF) energy delivery, may be used to reshape cartilage and/or other structures in the nose and/or to change other properties of tissues, such as nerves underlying the nasal mucosa lining the walls of the nasal cavity. The treatments may address any of a wide variety of conditions, just two examples of which are nasal valve insufficiency (which may cause difficulty breathing through the nose) and chronic or allergic rhinitis. Examples of nasal tissue treatment devices, systems and methods patented by the applicant include U.S. Pat. Nos. 8,936,594; 8,986,301; 9,072,597; 9,179,964; 9,179,967; 9,237,924; 9,415,194; 9,433,463; 9,452,010; 9,486,278; 9,687,296; 9,788,886; 9,801,752; 9.888.957; 9,913,682; 9,943,361; 10,028,780; 10,265,115; 10,335,221; 10,376,300; 10,398,489; 10,456,185; 10,456,186; 10,470,814, 10,485,603; 10,603,059; 10,631,925; 10,722,282; 10,779,873; 10,864,035; 10,932,853; 11,033,318; 11,096,738; 11,116,566; 11,241,271; 11,304,746; and 11,457,971, all of which are hereby incorporated by reference in this application, and all of which may be referred to collectively herein as the “Incorporated References.”

Rhinitis, or inflammation and swelling of the mucous membrane of the nose, causes symptoms such as runny nose, itchy nose, post-nasal drip, and congestion. Chronic and allergic rhinitis affect tens of millions of patients every year in the United States alone-one of the most common reasons for patients to visit physicians. Typically, rhinitis is treated with medications, such as nasal sprays and allergy shots. Unfortunately, these medications are temporary and are associated with multiple side effects. Surgical options are limited and invasive, typically involving resection of the vidian nerve.

The human nasal cavity is an area that extends from inside the two nostrils at the front of the nose to the junction of the nasal cavity with the back of the throat. The nasal septum is a wall of cartilage and bone that separates an anterior portion of the nasal cavity into the two nostrils. The lateral walls and septum of the nasal cavity are formed mainly of cartilage and bone, covered with a mucous membrane (or “nasal mucosa”). Other tissues also reside below the nasal mucosa, such as small blood vessels and nerves. Tissue underlying the nasal mucosa is sometimes referred to generally as submucosal tissue. One of the functions of mucosal tissue is to produce mucus, which helps protect, moisten, and clear debris from the nasal cavities. Nerves underlying the nasal mucosa are generally responsible for sensory function-feeling and smelling—and for activating the mucus-producing cells in the nasal mucosa to produce mucus.

Three bony structures covered in mucosal tissue, called the superior, middle, and inferior nasal turbinates, extend inwardly from each of the two lateral walls of the nasal cavity. FIG. 1A shows the right lateral wall of the nasal cavity, including the superior turbinate ST, superior meatus SM, a cut away middle turbinate MT, middle meatus MM, inferior turbinate IT, and inferior meatus IM. The downwardly curved edge of each turbinate defines a passageway below each turbinate, known as a meatus. The inferior meatus is located beneath the inferior turbinate, the middle meatus is located beneath the middle turbinate, and the superior meatus is located beneath the superior turbinate.

The turbinates are autonomically innervated by nerves arising from the Vidian nerve, which contains sympathetic and parasympathetic afferent nerve fibers that can modulate the function of the turbinates to either increase (parasympathetic) or decrease (sympathetic) activity of the submucosal layer. It has also been determined that one or more of the turbinates may also be innervated by the anterior ethmoidal nerve, which is a branch of the nasociliary nerve, which in turn is a branch of the ophthalmic nerve.

Turbinate reduction surgery is one surgical treatment for chronic or allergic rhinitis. This procedure is relatively invasive, however, and it typically has only temporary effects and can result in complications, such as mucosal sloughing, acute pain and swelling, and bone damage.

Another treatment for chronic rhinitis that was tried in the early 1960s through the 1970s was to transect (cut) the vidian nerve. Although the procedure was quite effective in treating chronic rhinitis, it was largely abandoned in the 1980s, primarily because it often results in dry eye, because autonomic fibers in the vidian nerve innervate the lacrimal glands.

Another surgical treatment method for chronic or allergic rhinitis, posterior nasal neurectomy, was initially developed in the late 1990s. Posterior nasal neurectomy involves cutting or cauterizing nerve tissue located in or near the sphenopalatine foramen, an opening in each of the two the lateral walls of the nasal cavity, where the sphenopalatine ganglion is located and out of which multiple nerves branch. Nerve fibers that pass through the sphenopalatine foramen branch into the middle and inferior turbinates and are distributed around the mucosal layer of the nasal cavity. Therefore, selective neurectomy at the foramen enabled physicians to more frequently avoid at least some surgical complications, such as dry eye. Still, cutting or cauterizing nerves at the sphenopalatine foramen is a delicate procedure in a difficult posterior location of the nasal cavity to reach and to see, and destroying nerves in that area may result in unwanted side effects.

Due to the large numbers of chronic rhinitis patients and the invasiveness and side effects of surgical procedures, it would be desirable to have improved devices, systems, and methods for treating chronic and allergic rhinitis. Ideally, such devices, systems, and methods would have longer term effects than medications and be less invasive than currently available surgical techniques. At least some of these objectives will be addressed in this application.

### BRIEF SUMMARY

This application describes various aspects and embodiments of a device, system, and method for treating nasal airway tissue. More particularly, the system includes: (1) a console with a radiofrequency (RF) electrosurgical generator and a display; and (2) a stylus coupled to the console via a cable. In alternative embodiments, the system may be configured to deliver another type of energy rather than RF, such as but not limited to heat, laser, microwave, cryogenic cooling, DC current, ultrasound, or chemical energy. The energy delivery system may be designed and used to perform different types of nasal airway tissue treatments. For example, and as further described in the Incorporated References, in one embodiment the system may be used to reshape, reconfigure and/or change another property of tissue (such as but not limited to cartilage) in or near a nasal valve area within the nose to enhance nasal breathing. In another embodiment, the system may be used to treat soft tissue in a more posterior portion of the nasal airway to treat chronic rhinitis, allergic rhinitis, post-nasal drip and/or chronic cough. Tissues treated in such a procedure may include nasal nerves, such as but not limited to an anterior ethmoidal nerve, a nasociliary nerve, any branch of the ophthalmic nerve, a posterior nasal nerve, the vidian nerve or any branch thereof, and/or the sphenopalatine ganglion or any branch thereof. Additionally or alternatively, the devices, systems, and methods may be used to treat submucosal tissue, mucosal tissue, goblet cells and/or any other nasal airway tissue. Although the following description focuses primarily on treating nerves in the nasal cavity to treat chronic rhinitis, allergic rhinitis, post-nasal drip and/or chronic cough, the devices, systems, and methods described herein may alternatively be used to perform any suitable procedure in a nasal airway, including but not limited to all procedures described in the Incorporated References.

### BRIEF DESCRIPTION OF THE DRAWINGS

FIG. 1A is a sagittal view of a human nasal cavity, illustrating the lateral wall and nasal nerves;

FIG. 1B is a sagittal view of a human nasal cavity, illustrating the lateral wall and the anterior ethmoidal nerve;

FIG. 2 is a front perspective view of an RF electrosurgical system for treating nasal airway tissue, according to one embodiment;

FIG. 3 is a close-up perspective view of the stylus, cable and connector of the system of FIG. 2;

FIGS. 4A and 4B are side and top perspective views, respectively, of a distal tip (or “treatment element”) of the stylus of FIGS. 2 and 3;

FIG. 5 is a perspective view of the console of FIG. 2, shown connected to an on/off foot pedal and a power cord, according to one embodiment;

FIG. 6 is a flow chart illustrating a method for treating nasal airway tissue using a system such as that shown in FIGS. 2-5, according to one embodiment;

FIG. 7 is a top view of the stylus of FIGS. 2-4B, illustrating a method for bending the shaft of the stylus, according to one embodiment;

FIG. 8 is a perspective view of the stylus of FIGS. 2-4B and 7, illustrating an indicator on the handle showing the direction of treatment, according to one embodiment;

FIG. 9 is a sagittal view of a human nasal cavity, illustrating a method for treating the posterior nasal nerve with the stylus described herein, according to one embodiment;

FIGS. 10A and 10B illustrate a method for addressing, contacting and treating nasal tissue, according to one embodiment;

FIG. 11 illustrates an improper treatment contact with nasal mucosa;

FIG. 12 is a perspective view of an electrosurgical system, according to an alternative embodiment;

FIG. 13 is a rear perspective view of the console of the electrosurgical system of FIG. 12;

FIGS. 14A-14C are top, front and side views, respectively, of the console shown in FIGS. 12 and 13;

FIG. 15 is a screen shot of a standby screen display that may be displayed on an electrosurgical console, according to one embodiment;

FIG. 16 is a screen shot of a default main screen display on an electrosurgical console, as it appears before the electrosurgery treatment has started, according to one embodiment;

FIG. 17 is a screen shot of a default main screen display on an electrosurgical console, as it appears during an RF delivery stage of an electrosurgery treatment, according to one embodiment;

FIG. 18 is a screen shot of a default main screen display on an electrosurgical console, as it appears during a cooling stage of an electrosurgery treatment, according to one embodiment;

FIG. 19 is a screen shot of a custom treatment settings screen display on an electrosurgical console, as it appears during a cooling stage of an electrosurgery treatment, according to one embodiment;

FIG. 20 is a screen shot of a fault screen display on an electrosurgical console, according to one embodiment;

FIG. 21 is a screen shot of an error screen display on an electrosurgical console, according to one embodiment;

FIG. 22 is a screen shot of a settings screen display on an electrosurgical console, according to one embodiment;

FIG. 23 is a flow diagram of a method of performing an electrosurgical treatment, using a console as described in the present application, according to one embodiment;

FIG. 24A-24C illustrate a method of using a shaft bending tool to bend a stylus, according to one embodiment;

FIG. 25A-25C illustrate a method of using a different shaft bending tool to bend a stylus, according to an alternative embodiment;

FIG. 26 is a perspective view of a nasal airway tissue treatment stylus with two bends in the shaft and bend markers on the shaft, according to one embodiment;

FIGS. 27A and 27B are side and front views, respectively, of a distal end of a nasal airway tissue treatment stylus, according to an alternative embodiment;

FIG. 28 is a front view of a distal end of a nasal airway tissue treatment stylus, according to an alternative embodiment;

FIGS. 29A and 29B are perspective and front views, respectively, of an alternative embodiment of a nasal airway tissue treatment stylus that includes an expandable wire electrode component;

FIGS. 29C and 29D are perspective views of the stylus of FIGS. 29A and 29B, illustrating operation of the expandable wire electrode component;

FIG. 30 is perspective view of another alternative embodiment of a nasal airway tissue treatment stylus that includes an expandable cryotherapy balloon;

FIGS. 31A and 31B are perspective and front views, respectively, of another alternative embodiment of a nasal airway tissue treatment stylus that includes an expandable wire electrode component that advances out of the distal tip of the device; and

FIG. 32 is a sagittal cross-section of a nasal cavity, illustrating an anterior ethmoidal nerve and a treatment device in position to deliver energy to treat the nerve.

### DETAILED DESCRIPTION

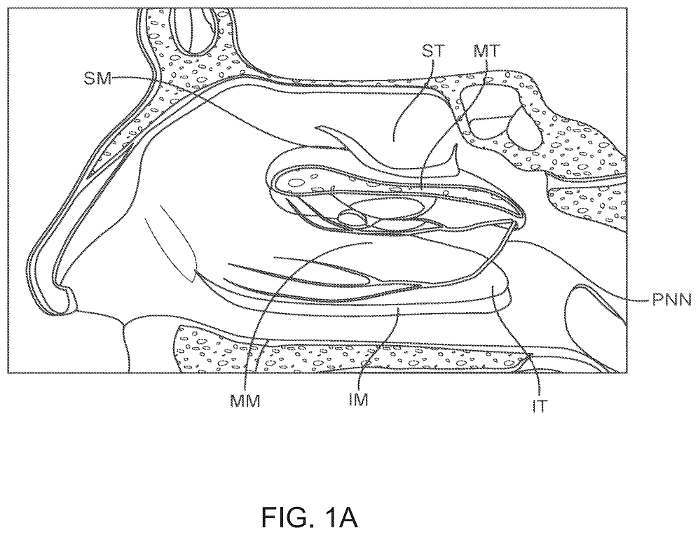

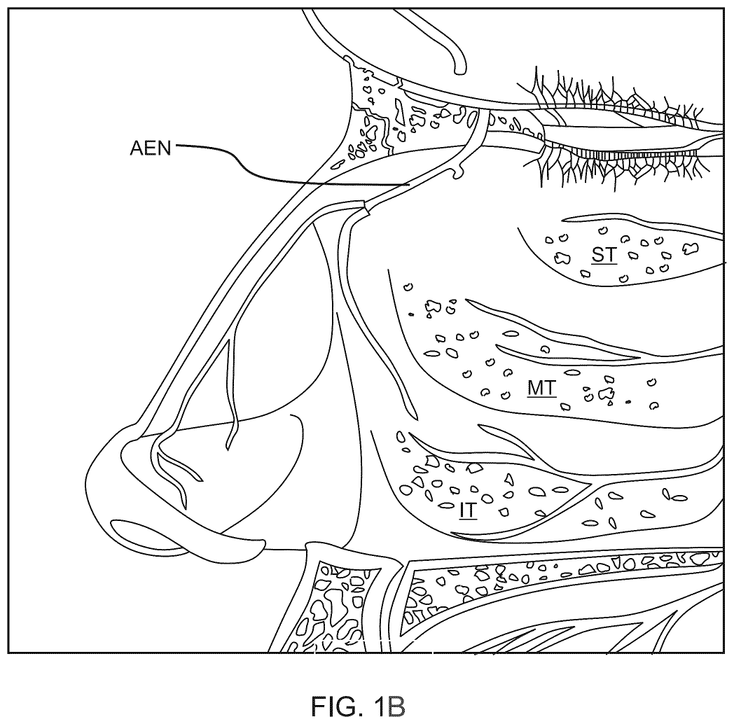

Referring to FIG. 1A, a sagittal cross-section of a human nasal cavity is shown. In this cross-sectional view, the middle turbinate MT has been removed, to show the path of the posterior nasal nerve PNN. Also shown are the superior turbinate ST, superior meatus SM, middle meatus MM, inferior turbinate IT, and inferior meatus IM.

FIG. 1B is another sagittal cross-section of a human nasal cavity. This figure illustrates the anatomical location of the anterior ethmoidal nerve AEN and its branches, in relation to the superior turbinate ST, middle turbinate MT, and inferior turbinate IT. In one embodiment, an energy delivery device may be placed in contact with nasal mucosa overlying the anterior ethmoidal nerve AEN and/or one or more of its branches, and energy may be delivered to the nerve to ablate or otherwise modify the nerve, thus treating chronic or allergy rhinitis and related symptoms, such as post-nasal drip and chronic cough. As mentioned above, the energy delivered may be any suitable form of energy, such as but not limited to radiofrequency (RF), heat, laser, microwave, cryogenic (i.e., removal of energy), DC current, ultrasound, or chemical energy. In various embodiments, a treatment may be directed at one nerve, such as the anterior ethmoidal nerve AEN, or at a combination of nerves, such as the anterior ethmoidal nerve AEN and the posterior nasal nerve PNN. A single treatment in a patient may involve one energy delivery from one device in a first nostril, followed by one energy delivery from the same device or another device per nostril. Alternatively, a single treatment in a patient may involve multiple energy deliveries in each nostril, with a physician moving a treatment device to different areas in the nasal cavity to address different nerves and/or different portions of the same nerve. Each delivery of energy may take as little as a few seconds up to several minutes.

Although the systems, devices and methods described herein may be used to treat other nerves and other tissues in the nasal cavity, the posterior nasal nerve and the anterior ethmoidal nerve are two primary nerve tissue targets for treating chronic rhinitis, allergic rhinitis, post-nasal drip and/or chronic cough. For the purposes of this application, the term “rhinitis” is used as an inclusive term to describe chronic rhinitis, allergic rhinitis, post-nasal drip, chronic cough, any combination thereof, and any symptoms that stem therefrom. In an actual patient, the PNN, anterior ethmoidal nerve, and other nasal nerves lie under the nasal mucosa and thus are not visible or on the surface, as they are in FIGS. 1A and 1B. This anatomical drawing is provided for illustration purposes only.

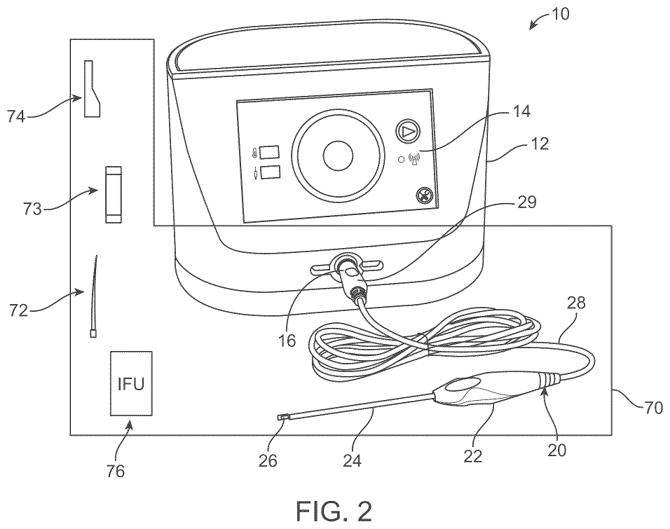

Referring to FIG. 2, in one embodiment, a nasal airway tissue treatment system 10 includes two primary components: a console 12 and a radiofrequency stylus 20 (or simply “stylus”). The console 12 includes a display 14, an RF generator and electronics (inside the console 12 and thus not visible), and an outlet 16 into which the stylus 20 is plugged. The stylus 20 includes a handle 22, a shaft 24, a distal tip 26 (also referred to as a “treatment element”), a cable 28 and a connector 29 for connecting the stylus 20 with the outlet 16. Many features of the console 12 and the system 10 in general are described further below, in U.S. patent application Ser. No. 16/668,678, and in the other Incorporated References.

The console 12 is a reusable device, which is designed and intended for use with multiple patients. The stylus 20, on the other hand, is a single-patient, single-use, disposable device. In some embodiments, the stylus 20 may be provided as part of a stylus kit 70, which may include a curved anesthesia needle 72, a packet of conductive gel 73, a shaft bending tool 74 and/or instructions for use 76 (or “IFU”). All of these kit components are optional, and any embodiment of the stylus kit 70 may include fewer items or additional items, without departing from the scope of the invention. In some embodiments, the stylus 20 may be provided by itself for use with the console 12. The stylus 20 may be used for multiple treatments on the same patient at the same time—for example multiple treatment areas in a nostril and/or treatment of both nostrils—and then is disposed of after use on that patient. In an alternative embodiment, the stylus 20 may be sterilizable and reusable.

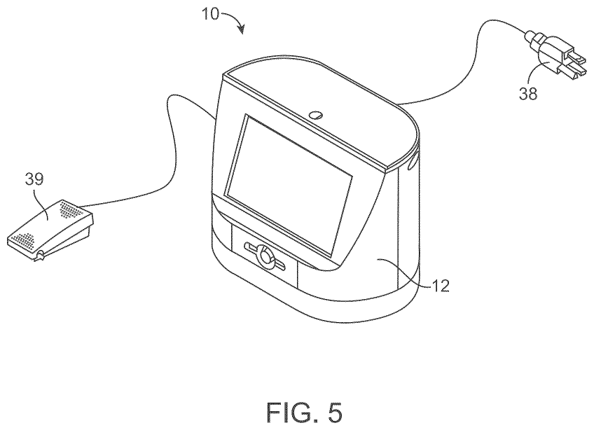

In some embodiments, the nasal airway tissue treatment system 10 may be provided with one or more additional components or accessories. For example, and as shown in FIG. 5, the system 10 typically includes a power cord 38 and a foot switch 39, both of which attach to the console 12. As another example, the stylus 20 illustrated in FIG. 2 has a relatively long shaft 24, configured for use in the posterior portion of the nasal cavity to treat nerves to treat rhinitis. In one embodiment, one or more additional styluses may be provided with the system 10, for performing other procedures. For example, a second stylus with a shorter shaft for addressing tissue in the nasal valve, closer to the front of the nose, may be provided. The curved anesthesia needle 72 for injecting anesthetic into the nasal mucosa is another optional component. The curved needle 72 is designed to be stiffer than a spinal needle and is curved to allow a physician to easily access and inject anesthesia into an area near the back of the nasal cavity. The curved needle 72 can also be reinforced to prevent bending or bowing during injection. The system 10 may also be provided with one or more conductive gel packets 73, for application to the distal tip 26 of the stylus, to enhance contact of the distal tip 26 with nasal mucosa. The bending tool 74 will be described further below and may be used by the physician to bend the shaft 24 of the stylus 20 in one or more locations. Any other suitable accessories or components may be added to the treatment system 10, according to alternative embodiments.

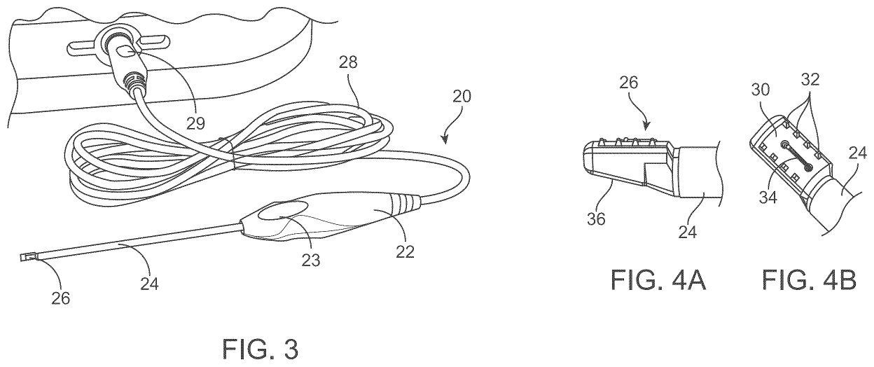

FIG. 3 is a more detailed view of the RF stylus 20. In this embodiment, the handle 22 includes an oval depression 23, which faces in the same direction as the treatment surface of the distal tip 26. The oval depression 23 allows the physician user to know the orientation of the treatment surface at all times and may also be used as a finger or thumb rest. In some embodiments, an on/off button may be positioned where oval depression 23 is illustrated on handle 22. This on/off button may be used to activate the stylus and thus provide RF energy to the electrodes at the distal tip 26. In some embodiments, the physician may use the handle on/off button as an alternative to a foot pedal for this purpose. The stylus shaft 24 may have any length suitable for reaching the posterior portion of the nasal cavity where the posterior nasal nerves reside. In one embodiment, for example, the shaft 24 is approximately 3.75 inches long, from its connection to the distal tip 26 at one end to its connection with the handle 22 at the opposite end. In alternative embodiments, the shaft 24 may have different lengths, for example between about 3 inches and about 4 inches in embodiments designed for addressing a posterior portion of the nasal cavity, and between about 1 inch and about 3 inches in embodiments designed for addressing an anterior portion of the nasal cavity, such as the nasal valve. The shaft 24 may also have any suitable width (or “diameter”). For example, in some embodiments, the shaft may have a width of about 4 mm to about 5 mm. The shaft 24 will be described in further detail below.

Referring now to FIGS. 4A and 4B, the distal tip 26 is shown in detail. The distal tip 26 includes a treatment surface 30 (or “tip face”), two rows of four bipolar RF electrodes 32, and a thermocouple 34 between the electrodes 32. As illustrated in FIG. 4A, the bottom surface 36 of the distal tip 26 may be tapered or slanted, to give the distal end of the distal tip 26 a narrower profile to facilitate insertion and advancement of the distal tip 26 into the nostril and to the posterior portion of the nasal cavity. The treatment surface 30 is made of a non-conductive material (e.g., plastic) and may have a slightly convex shape, as illustrated, or may alternatively be flat, concave, or more convex than the shown in FIG. 4B. The slightly convex shape may help ensure contact of the electrodes 32 and the thermocouple 34 with mucosal tissue during use. In alternative embodiments, the thermocouple 34 may be removed or replaced by a different type (or shape or number) of temperature sensor(s). The thermocouple 34 is used to measure temperature of the mucosa and provide the measurements to a processor in the console 12, which can in turn automatically adjust whether and how much RF energy is delivered to the stylus 20. In some embodiments, the distal tip 26 or a portion thereof may be translucent, to facilitate visualization of target tissue and/or nasal mucosa.

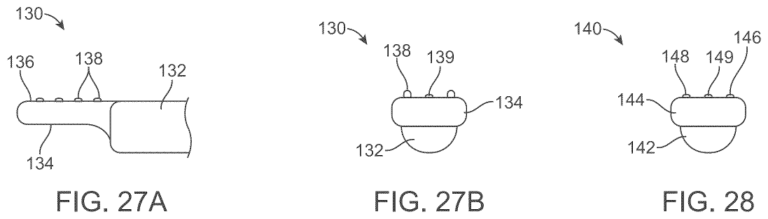

FIGS. 27A and 27B illustrate in side and front views, respectively, a distal portion of an alternative embodiment of a nasal airway tissue treatment stylus 130. In this embodiment, the height or thickness of the distal tip 134 is thinner than in the previously described embodiment and thinner than the shaft 132 of the stylus 130. The distal tip 134 includes a treatment surface 136, four pairs of bipolar RF electrodes 138, and a thermocouple 139. The thinner distal tip 134 may facilitate passage of the tip 134 through a nostril and around nasal cavity anatomy for positioning at a treatment site in a posterior portion of the nasal cavity. In all other respects, the distal tip 134 may include any of the features of the embodiment described above.

FIG. 28 is a front view of yet another embodiment of a nasal airway tissue treatment device 140. This embodiment also includes a shaft 142 and a distal tip 144, which in turn includes a treatment surface 146, four pairs of bipolar RF electrodes 148 and a thermocouple 149. In this embodiment, however, the electrodes and thermocouple are flat, rather that rounded or pointed. They protrude only slightly from the treatment surface 146. This further helps reduce the overall height or thickness of the distal tip 144 and thus makes it easier to advance the tip 144 through the nostril and nasal cavity. In some embodiments, the electrodes and thermocouple may even be completely flush with the treatment surface.

FIG. 5 illustrates the console 12 of the nasal airway tissue treatment system 10, along with a power cord 38 and a foot switch 39, both of which plug into the back of the console 10. The foot switch 39 is used by the physician to activate the stylus 20 during a procedure. In alternative embodiments, the handle 22 of the stylus 20 may include an on/off button or switch, in addition to or as an alternative to the foot switch 39.

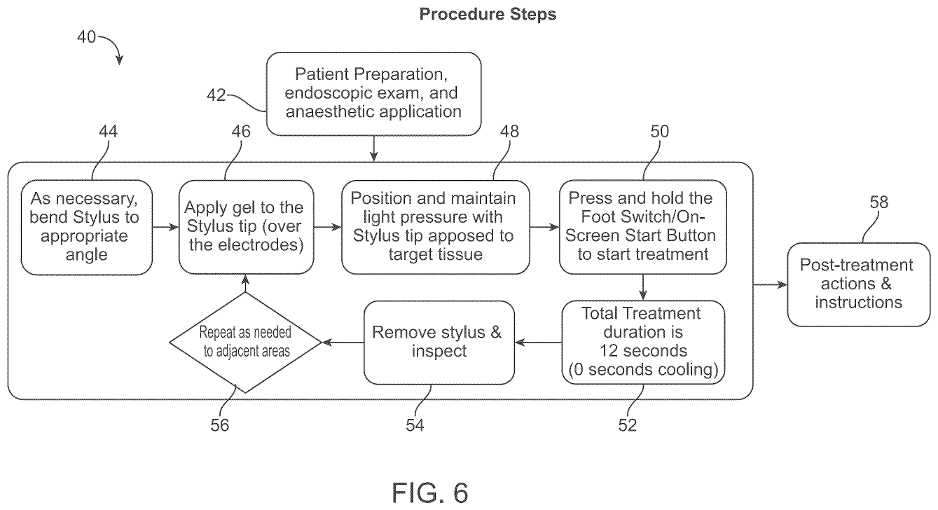

FIG. 6 illustrates one embodiment of a method 40 for using the treatment system 10 to treat nerve tissue (and/or other tissues) in the nasal cavity to treat rhinitis. In this embodiment, the method 40 includes the steps of: preparing the patient 42, such as with local anesthesia and an endoscopic examination of the nasal cavity; bending the stylus 44, if desired (explained further below); applying conductive gel to the stylus distal tip 46, over the electrodes; advancing the distal tip of the stylus into the nostril and contacting it with the treatment area 48, then maintaining light pressure with the distal tip against the treatment tissue; pressing and holding down the foot switch 50 to start the treatment; continuing to hold down the foot switch for twelve seconds 52 (the treatment time in this embodiment); removing and inspecting the distal tip 54; and repeating 56 steps 46, 48, 50 and 52 as necessary, to treat additional treatment areas. In one embodiment, for example, it is recommended that at least three, twelve-second treatments are performed along the course of the posterior nasal nerve. Any other number of treatments may be performed, however, in alternative embodiments. Additionally, each treatment may last a different period of time than 12 seconds, such as between 10 seconds and 60 seconds in various embodiments. When a treatment on a patient is complete, a final step may be to provide post-treatment actions and instructions to the patient 58.

The method may be repeated for as many treatment areas as desired. In some embodiments, the stylus 20, the console 12 or both may be configured to allow only a certain number of treatments for any given stylus 20. This may help prevent reuse of the stylus 20 on multiple patients or overtreatment of any one patient. For example, in one embodiment, the stylus 20 may only be able to deliver sixteen 12-second treatments. In other embodiments, the stylus 20 may be capable of delivering ten to thirty 12-second treatments, for example. In yet other embodiments, the stylus 20 may be capable of delivering any number of treatments, but the console 12 is able to identify each stylus 20 and count or identify how many treatments have been applied with that stylus 20. The console 12 may be configured to shut down or simply not deliver RF energy to a stylus 20 that has reached its maximum number of allowed treatments. In other embodiments, a single stylus 20 may be used with the console 12 to deliver as many treatments on one patient as desired, but once the treatment on that patient is completed, the stylus 20 is rendered inoperable for use with any additional patient(s). Inoperability may be conferred by a computer chip in the handle 22 of the stylus 20, or alternatively the console 12 may destroy or alter a portion of the stylus 20 when the stylus 20 is unplugged from the console 12 or at some other point at the end of a treatment.

In some embodiments, the nasal airway tissue treatment stylus 20 may be used for treating several different types of target tissue in one patient. Before treating posterior nasal nerve tissue, after treating that tissue, or both, one or more additional tissues may be treated. Such tissues include mucosa, nerves and/or other tissue of any one of the nasal turbinates, nasal swell bodies, the nasal septum, and mucus producing cells anywhere in the nasal cavity. Therefore, the treatment method illustrated in FIG. 6 may be added to by treating any one or more of these additional tissues. Physicians may often treat the inferior and/or middle turbinate before or after proceeding with the steps in FIG. 6 to treat posterior nasal nerve tissue.

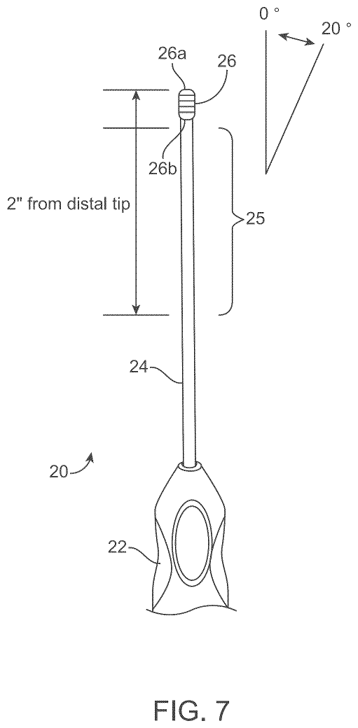

Referring next to FIG. 7, a portion of the stylus 20 is shown, illustrating features of the shaft 24. In this embodiment, the shaft 24 is malleable, so that it can be bent by a physician user and retain the bent shape during use. This may be especially advantageous, for example, in helping the physician advance the distal tip 26 of the device around anatomical structures to a posterior target area in the nasal cavity. As illustrated in FIG. 7, the distal tip 26 has a distal edge 26a and a proximal edge 26b. Just proximal to the proximal edge 26b of the distal tip 26 is a bend section 25 of the shaft 24. The bend section 25 is approximately 2 inches in length in this embodiment, and it is the section of the shaft that is recommended for bending. Physician users may be instructed, for example with instructions for use provided with the system 10, to bend the shaft 24 only within the bend section 25 and not too close to the distal tip 26. This may help prevent weakness at the areas where the shaft 24 is connected to the distal tip 26 and the handle 22. In the embodiment shown, the entire shaft 24 is made of a malleable material, but in alternative embodiments only a portion of the shaft 24 might be malleable, such as the bend section 25.

The shaft 24 may be manually bent by the physician to the appropriate bend angle. While bending, the physician should support the stylus 20 by the shaft 24, not by the handle 22 or the distal tip 26. The bend should be formed in the orientation the electrodes 32 are facing. The shaft 24 may be bent to any suitable angle. In one embodiment, however, it is recommended that the shaft 24 only be bent to a maximum of approximately 20 degrees away from the longitudinal axis of the stylus 20. Again, this limit on bending may help maintain the structural integrity of the stylus 20. Bending the shaft 24 at all is entirely optional, and some or even all physicians might decide not to bend the shaft 24 at all. In alternative embodiments, the shaft 24 might be rigid and not malleable. In general, all parts of the stylus 20, other than the electrodes 32 and the thermocouple 34, may be made of non-conductive materials, such as any suitable plastic or polymer.

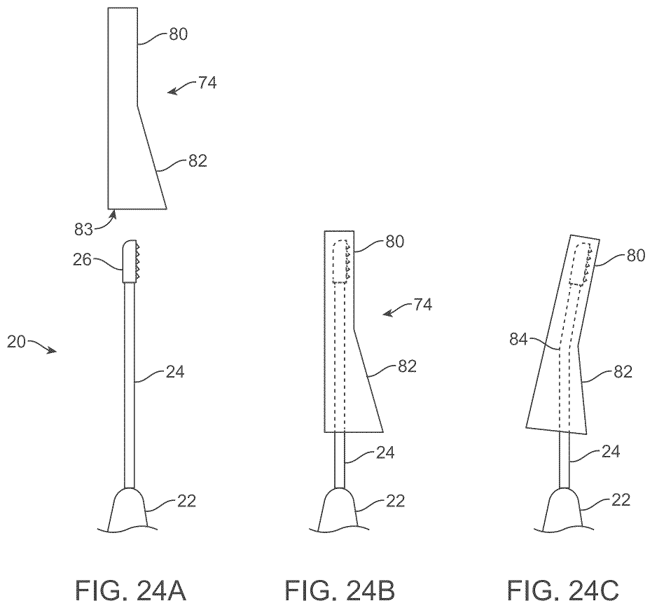

Referring to FIGS. 24A-24C, in some embodiments a bending tool 74 may be provided for bending the shaft 24 of the stylus 20. In this embodiment, the bending tool 74 includes a narrow distal portion 80, a wide proximal portion 82, and an opening 85 into which the distal tip 26 and a portion of the shaft 24 enter. As illustrated in FIG. 24B, the bending tool 74 may first be slid down onto the stylus 20. The bending tool 74 may then be tilted, as shown in FIG. 24C, to form a bend 84 in the shaft 24. The bending tool 74 may have any suitable length, to bend the shaft 24 in a desired location. For example, in some embodiments the bending tool 74 is designed to bend the shaft 24 somewhere between one third and one half of the length of the shaft measured from the handle 22. Alternative embodiments of the bending tool 74 may have any other suitable size, shape and configuration for bending a shaft 24 of a stylus 20.

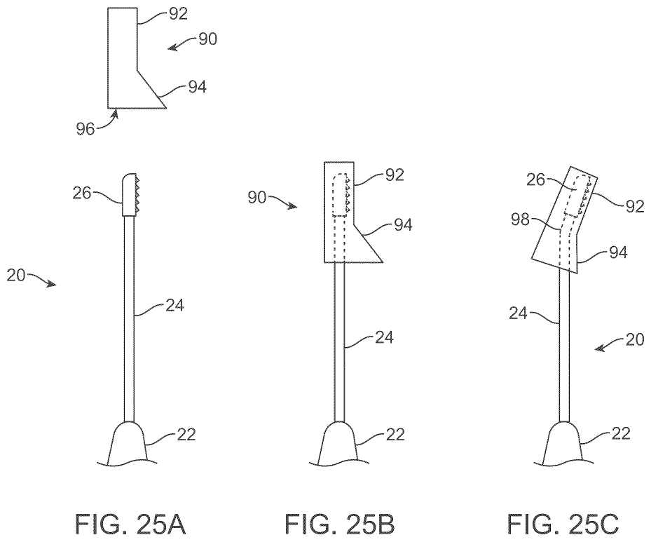

FIGS. 25A-25C illustrate the use of another embodiment of a shaft bending tool 90. In this embodiment, the shaft bending tool 90 includes a narrow distal end 92, a wide proximal end 94, and an opening 96, as with the previous embodiment. In this embodiment, however, the shaft bending tool 90 is significantly shorter than the previously illustrated embodiment. As illustrated in FIG. 25C, this shorter bending tool 90 creates a bend 98 in the shaft 24 that is much closer to the distal tip 26. Physicians may want to bend the shaft 24 of the stylus 20 in different locations. Thus, in some embodiments more than one size or shape of bending tool 74, 90 may be provided. A physician may select one bending tool 74, 90 to make one bend, or she may use two (or more) different bending tools 74, 90 to make multiple bends in the shaft 24.

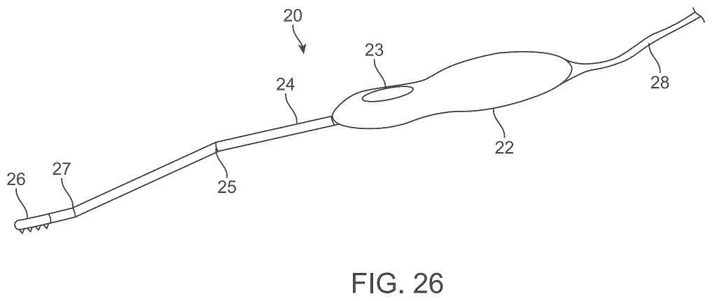

Referring to FIG. 26, as just mentioned, in some cases a physician may bend the shaft 24 of the stylus 20 at two different locations, forming for example a proximal bend and a distal bend. In some embodiments, the shaft 24 may include one or more proximal bend markers 25 and one or more distal bend markers 27. Bend markers 25, 27 may be located at any location along the length of the shaft 24 and may be made with paint, etched into the surface, or by any other means. In the illustrated embodiment, two bends are made to give the shaft 24 a bayonet shape, as desired by some physicians to navigate around anatomical structures in the nasal cavity while still positioning the treatment surface of the distal tip 26 in full contact with nasal mucosa on the lateral wall of the nasal cavity. It bears repeating that in various alternative embodiments, the shaft 24 may include any number of bend markers 25, 27 at any locations and/or the shaft 24 may be bent (with or without markers) at any suitable location or at multiple locations.

In various embodiments, the shaft 24 may be either more or less malleable, depending on the desired stiffness versus bendability of the shaft 24. In some embodiments, only certain portions of the shaft, which are designed to be bent, are malleable, while others are stiff. Or certain portions may be more malleable than others. More malleable sections may have a thinner wall than less malleable sections and/or the shaft 24 may be made of different materials in different sections. The latter is likely to complicate manufacturing and increase expense, however, so in at least some embodiments the shaft 24 is made of one piece of material, such as a metal hypotube. In such cases, differences in malleability may be achieved via differences in wall thickness.

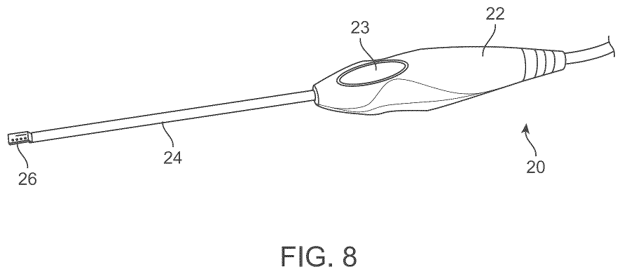

FIG. 8 is another illustration of the stylus 20, showing how the oval depression 23 on the top of the handle 22 aligns with the treatment surface 30 of the distal tip 26. This allows the physician to always know what direction the treatment surface 30 is facing. The oval depression 23 is configured to provide an ergonomic location for placement of the physician's thumb, but depressions of different shapes may be used in different embodiments. Other alternative embodiments may use any other suitable directional indicator on the handle 22 and/or a proximal portion of the shaft 24, to show direction of the distal tip 26.

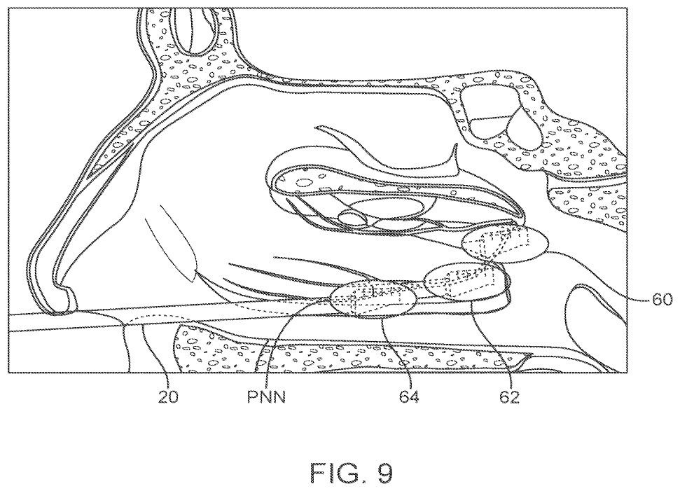

FIG. 9 is the same sagittal view of the nasal cavity as in FIG. 1, with the additional illustration of three treatment areas for treating the nasal cavity with the stylus 20. In this embodiment, the stylus 20 has been used to deliver RF energy to a first target location 60, a second target location 62 and a third target location 64, all of which fall along the path of the posterior nasal nerve PNN. Additional treatments may optionally be provided in the same general area and/or in different areas of the nasal cavity. For example, some physicians may use the stylus 20 to treat a nasal septum, a septal swell body, an inferior turbinate and/or other soft tissue(s) in the nose, all as part of the same treatment on one patient. FIG. 9 is thus provided for illustrative purposes only.

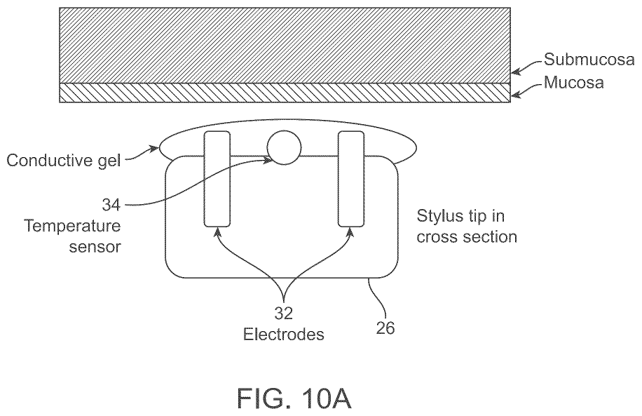

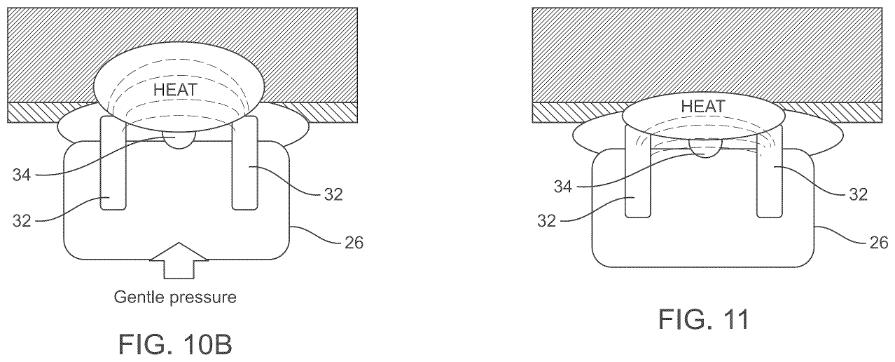

Referring to FIGS. 10A and 10B, two steps of the method for using the nasal tissue treatment system 10 are illustrated diagrammatically. In FIG. 10A, the distal tip 26 of the stylus 20 is shown in an end-on, cross-sectional view, showing two electrodes 32 and the temperature sensor 34. The distal tip 26 has conductive gel on its treatment surface 30 and is in position within the nostril, near the mucosa. Next, as illustrated in FIG. 10B, the physician contacts the mucosa with the distal tip 26, applies gentle pressure against the mucosa, and presses the foot pedal to activate the stylus 20 and deliver RF energy from one set of electrodes 32, through the mucosa, to target tissue(s) in the submucosa, and back to a second set of electrodes 32. This process generates heat and treats the target submucosal tissues, such as the posterior nasal nerve and/or other nerves. Again, the treatment may be timed by the console 12, which may automatically stop the delivery of RF energy after a preset time, such as 12 seconds in one embodiment. The thermocouple 34 measures the temperature of the mucosa it is contacting, and the system 10 uses this measurement to automatically shut off the system 10 if a temperature is too high or otherwise outside of a preset range of acceptable temperatures.

FIG. 11 illustrates the distal tip 26 in a similar position as shown in FIG. 10B, but in this case the physician is not applying gentle pressure to the mucosa with the distal tip 26. As illustrated, it is possible that without application of pressure, the applied RF energy will not penetrate the submucosa to reach the desired target tissue. In some embodiments, the stylus 20 is designed to determine whether adequate pressure is being applied and to only activate the electrodes 32 when the pressure is applied.

According to various embodiments, the console 12 of the treatment system 10 may include default settings and custom settings. Default settings may include, for example, a power output of 4 Watts, a treatment temperature of 60 degrees Celsius, and a treatment time of 12 seconds. Custom settings may allow a physician to customize settings. For example, such settings could provide for power of 3-5 Watts with an increment interval of 1 Watt, a treatment temperature of 50-70 degrees Celsius with an increment interval of 5 degrees Celsius, and a treatment time of 10-12 seconds with an increment interval of 2 seconds. These are merely examples, however, and should not be interpreted as limiting.

Anesthesia protocols for anesthetizing the patient's nasal cavity are largely up to the physician, and many different protocols are known to otolaryngologists. In some embodiments, it may be required or strongly recommended to inject anesthetic into the mucosa and/or submucosa in the target area(s), in order to help direct the RF current delivered by the stylus 20. This may be helpful in some embodiments, because fluid such as anesthetic is generally conductive for RF.

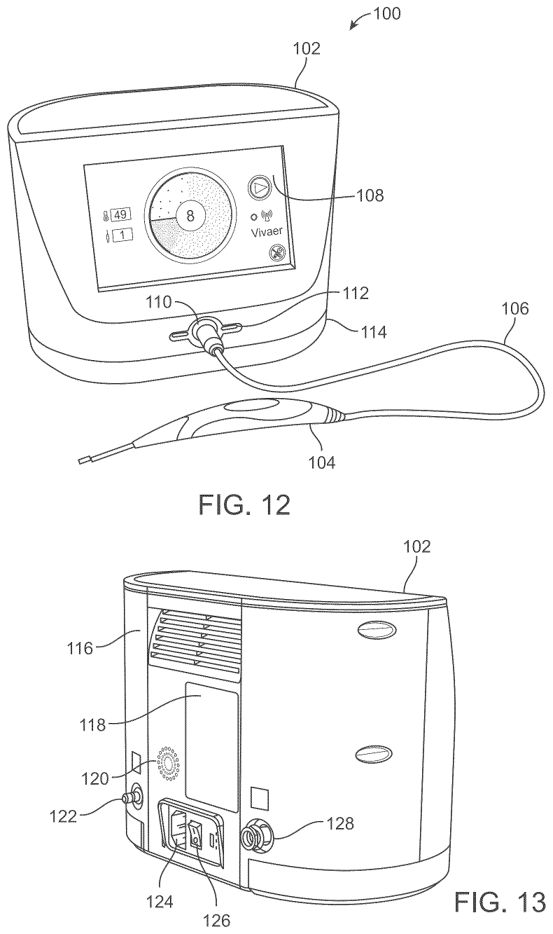

Referring to FIG. 12, another example of an RF electrosurgical system 100 is illustrated. In this embodiment, the stylus 104 has a shorter shaft, such as might be used in treating tissues in the nasal valve area. Thus, the treatment times and protocols, as well as the displays on the display screen 108 of the console 102, are designed for nasal valve treatment. These features may be adjusted/altered for treatment of nasal nerve(s) to treat rhinitis. As mentioned above, in alternative embodiments the electrosurgical system 100 may be modified to provide and deliver any other suitable type of energy, rather than RF. In this example, the RF electrosurgical system 100 includes a console 102, an RF delivery stylus 104, and a cable 106 connecting the stylus 104 to the console 102. The console 102 houses an RF generator, a processor and various electronics, none of which is visible in FIG. 1. The word “console,” in this disclosure, is meant to encompass the terms “generator,” “box” and any other commonly used terms to describe an electrosurgical system console or generator. The parts of the console 102 that are visible in FIG. 1 include a touch screen display 108, a stylus connection port 110, an “RF ON” indicator light 112, and a bottom ring 114. The touch screen display 108 serves as the main user interface for interacting with the console 102 and will be described further below. The stylus connection port 110 allows a connection end (or “connector”) of the stylus 104 to be plugged into it. The stylus connection port 110 is configured to accept only the connection end of the stylus 104 and will not accept or work with counterfeit or other devices. In some embodiments, electronics inside the console 102 may include a stylus identification safety feature that identifies the stylus 104 when it is plugged into the stylus connection port 110. Such a safety feature may, for example, automatically shut down (or disable powering on) the console 102, if a user tries to plug in a device other than the stylus 104.

The RF ON indicator light 112 indicates when RF energy is being delivered through the stylus connection port 110 to the stylus 104. The bottom ring 114, in this embodiment, lights up when the console 102 is powered on. This lighted ring 114 is an optional feature. Both the RF ON indicator light 112 and the lighted bottom ring 114 may have any color or colors of light. In one embodiment, for example, the RF ON indicator light 112 is blue, and the bottom ring 114 lights up with a white light. This is merely one example, however, and any suitable lighting configuration and combination of colors may be used in alternative embodiments.

FIG. 13 shows the back of the console 102. In this embodiment, the console 102 includes an air vent 116, a product label area 118, a speaker 120, an equipotential ground connection port 122, a main power connection port 124, a main power switch 126 and foot switch connection port 128. The equipotential ground connection port 122 is provided at the back of the device.

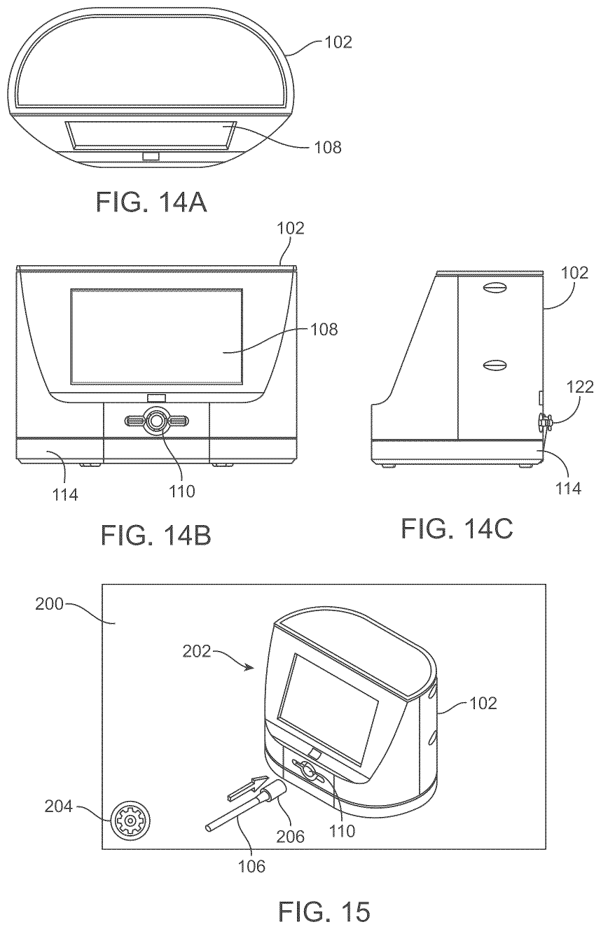

FIGS. 14A-14C are top, front and side views, respectively, of the console 102 shown in FIGS. 12 and 13.

Referring now to FIG. 15, a screen shot of one example of a standby image 200 that may be shown on the display screen 108 of the console 102 is illustrated. The standby image 200 may be displayed after the console 102 is powered on. In some embodiments, the console 102 may perform a power-on self-test before showing the standby image 200. The standby image includes a console image 202, a settings button 204 and an animated insert stylus image 206. The animated insert stylus image 206 shows the connector end of the stylus cable 106 moving toward the console stylus connection port 110. Thus, the display 200 graphically informs the user that the console 102 is ready for use, awaiting insertion of the connection end of the stylus cable 106 into the connection port 110.

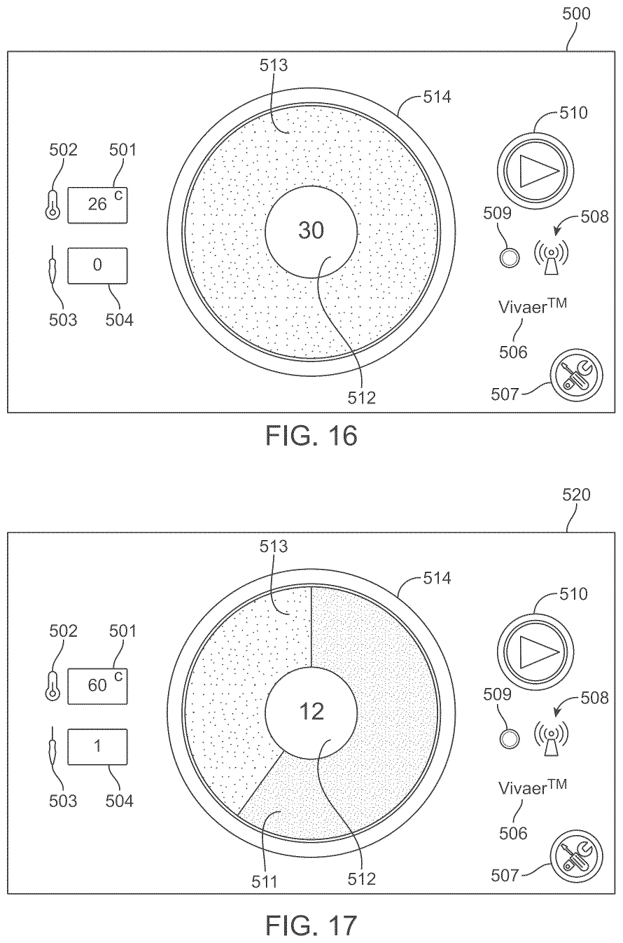

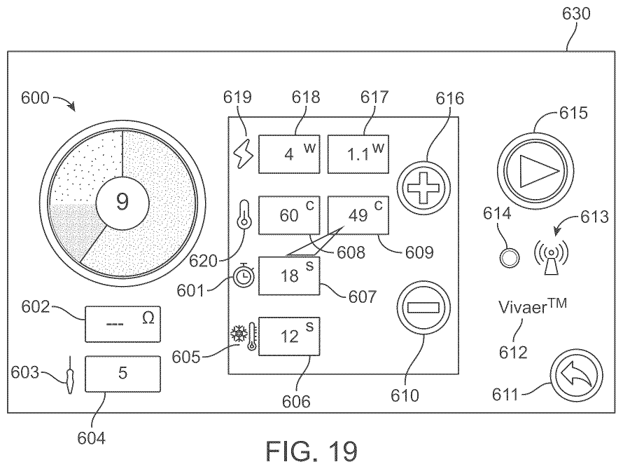

Referring now to FIG. 16, once the physician or other user has attached the stylus 104 to the console 102, the standby image of FIG. 4 may be replaced by a default main screen image 500. This main screen image 500 is what the user sees before the procedure has started. Under normal operating conditions, the user can select either default treatment settings or manual treatment settings. The default treatment settings are pre-loaded into the processor of the console 102 and do not require any additional settings inputs from the user. In some embodiments, it may be possible for the user to select from several sets of default settings. Operation under the default settings mode is described in relation to FIGS. 16-18, and operation under the custom settings mode is described in relation to FIG. 19.

FIG. 17 shows the default image 500 displayed on the display screen 108 of the console 102, once a valid stylus 104 is connected. In this embodiment, the default image includes a stylus temperature indicator 501, a temperature icon 502, a stylus icon 503, treatment number indicator 504, a stylus type connected indicator 506, a start/stop button 510, an RF ON indicator light 509, an RF icon 508, a custom treatment button 507 and a central, circular, graphical treatment progress display 514.

The graphical treatment progress display 514 has several portions, according to the embodiment shown in FIG. 16. First, there is a total treatment timer 512, which is displayed as a central circle with a counting down number, representing seconds (or alternatively minutes, or minutes and seconds) remaining in the current procedure. Here, for example, the treatment has not started yet, and the total treatment timer 512 shows a time of 30 seconds, thus indicating that at least the next tissue treatment will last 30 seconds total. Immediately surrounding the central circle total treatment timer 512 is a treatment time indication ring 513, which acts as an indicator of elapsed and remaining time in the procedure. In the default image 500 of FIG. 16, the treatment has not started, so the entire outer ring 513 is one initial color. The initial color of the ring 513 indicates time remaining in the treatment, which in this case takes up the entire ring 513.

Other indicators on the screen shot image 500 also show that the treatment has not yet started. For example, the RF ON indicator light 509 is not illuminated yet, because the console 102 is not yet delivering RF energy to the stylus 104. The treatment number indicator 504 shows that zero treatments have been performed with the stylus 104 that is currently plugged into the console 102. And the temperature indicator 501 shows a stylus temperature of 26 degrees Celsius. To begin a treatment, the physician user will touch the start/stop button 510 on the touchscreen 108.

Referring now to FIG. 17, a later screen shot 520 of the default settings screen is illustrated. At this stage, RF energy is being delivered from the console 102 to the stylus 104, as indicated by the RF ON indicator light 509. The total treatment timer 512 of the graphical treatment progress display 514 shows that 12 seconds remain in the treatment. The outer ring 513 now includes a darker RF energy delivery time portion 511, and the lighter remaining portion of the ring 513 indicates the portion of the total treatment time that is still remaining. As the RF energy delivery stage of the treatment begins and progresses, the darker RF energy delivery time portion 511 takes up more and more of the outer ring 513, moving in a clockwise direction. In other words, the RF energy deliver indicator 511 starts at zero, at the twelve o'clock position on the ring 513, and moves around the ring in a clockwise direction.

Other indicators that the treatment is in progress include the temperature indicator 501 showing a temperature of 60 degrees Celsius and the treatment number indicator 504 showing that this is the first treatment being performed with the stylus 104 currently plugged into the console 102.

In some embodiments, the console 102 may be activated, and RF energy delivered to the stylus 104 in either of two ways—the start/stop button 510 may be touched, or a foot pedal coupled with the console 102 may be depressed. The RF ON indicator 509 lights up when the console 102 is delivering RF power. The stylus type connected indicator 506 indicates what type of stylus 104 is connected to the console 102, which in the example shown is a Vivaer™ stylus (Aerin Medical, Inc., www.aerinmedical.com). This indicator 506 may be useful in embodiments where the console 102 is configured for use with multiple different types of styluses. The stylus temperature indicator 501 shows the actual temperature of the distal, treatment end of the stylus 104. The treatment number indicator 504 displays the number of the treatment currently being completed with the stylus 104 that is attached to the console 102. Finally, the custom treatment button 507 allows the user to customize one or more treatment parameters. Touching this button 507 will lead the user to a new display screen with different options. In alternative embodiments, the various icons and/or indicators on the default display 520 may be changed or moved. In some embodiments, one or more of the icons and/or indicators may be eliminated.

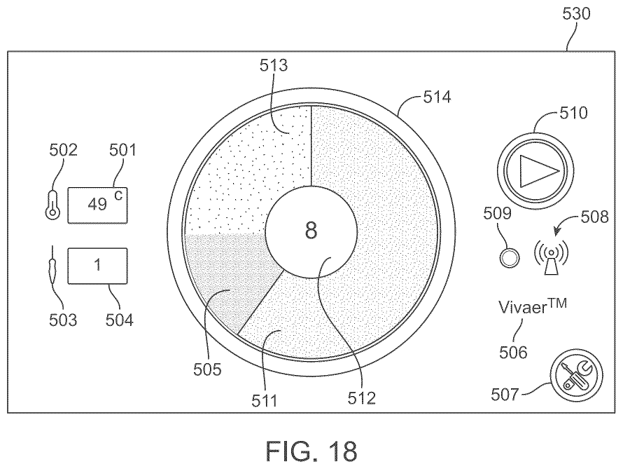

Referring now to FIG. 18, a third screen shot 530 of the default setting screen is illustrated. At a given amount of time into the procedure, the console 102 stops delivering RF energy to the stylus 104, and a cool down phase begins. The cool down phase is shown on the ring 513 as a differently colored or shaded segment 505, as compared to the RF ON segment 511, and it may be called a cool down timer indicator 505. The cool down timer indicator 505 moves around the ring in a clockwise direction until the procedure is complete. Any amount of time remaining in the procedure is indicated by the total treatment time remaining portion of the ring 513.

In the screen shot of the main screen image 530 shown in FIG. 18, the current stage of treatment is illustrated as follows: The central circle total treatment timer 512 shows there are eight seconds remaining in the treatment. The RF ON indicator segment 511 and the cool down timer segment 505 show that the RF delivery portion of the treatment is complete, because the cool down timer segment 505 has started. Since the total treatment time remaining portion of the ring 513 is still visible, this shows the user that the cool down phase is still in process. In a real life scenario, the cool down timer portion 505 would also be moving clockwise, thus easily telling the user what phase the treatment was in. In this case, the total treatment time portion of the ring 513 shows that approximately one fourth of the entire treatment time still remains. The temperature of 49 degrees Celsius in the temperature window also tells the user that the console 102 is in the cooling phase, since the temperature of the stylus 104 has decreased from 60 degrees. Finally, the RF ON indicator light 509 is not illuminated.

In various embodiments, any colors, shades, shapes, graphics and/or the like may be used for the various segments 511, 505 of the outer ring 513. In one embodiment, for example, the RF ON timer indicator 511 is navy blue, the cool down timer indicator 505 is gray, and the total treatment time remaining portion 513 is light blue. Any other colors may be used, however, in alternative embodiments. In another alternative embodiment, the entire ring 513 may be one color, and a line that acts as a timer may move clockwise around the ring 513, similar to a long hand on a clock. In a variation on such an embodiment, the color of the ring 513 behind the moving line may change. Thus, the ring 513 and the segments 511, 505 may have any suitable size, color scheme or configuration.

Additionally, the default (or custom) settings of the console 102 may have any suitable ranges and combinations for the various parameters of the console 102. For example, one timing default setting may have a total treatment time of 30 seconds, an RF ON time of 18 seconds, and a cooling time of 12 seconds. This is but one example, however, and any number of other time settings may alternatively be used. A default temperature may also be set for RF delivery, for example 60 degrees Celsius as the maximum temperature. Again, any suitable default settings may be set in various embodiments.

Referring now to FIG. 19, as just described, the physician or other user can choose to access a custom treatment screen 630 by touching the custom treatment button 507 on the initial default screen 500 (FIG. 16). In the embodiment shown, the custom treatment screen 630 includes a graphical treatment progress display 600, which is smaller but otherwise the same as the graphical treatment progress display 514 of the default screen 500. The custom treatment screen 630 also includes a timer icon 601, an impedance display 602, a stylus icon 603, a number of treatments indicator 604, a cooling icon 605, a set cooling time window 606, a set RF ON time window 607, a set temperature window 608, an actual temperature indicator 609, a down button 610, a back button 611, a stylus type indicator 612, an RF icon 613, an RF ON indicator 614, a start/stop button 615, an up button 616, an actual power delivery indicator 617, a set power window 618, an RF power icon 619 and a temperature icon 620. As with the previously described default screen 500, the icons and/or indicators of the custom treatment screen 630 may be moved, changed and/or eliminated, according to various alternative embodiments.

Through the custom treatment screen 630, the user can adjust the power (power window 618), temperature (temperature window 608), treatment time (RF on time window 607) and/or cool down time (cooling time window 606), by touching any one of the set windows and then touching the up button 616 and/or the down button 610 to adjust a given value. To set power, for example, the user may touch the power window 618 and then adjust the temperature by pressing the up button 616 or the down button 610. The console 102 may be configured to only allow adjustments within ranges. For example, the power on the console 102 may be selected at 3 W, 4 W or 5 W in one embodiment. Maximum stylus temperature may be selected in a range of 50 degrees Celsius to 70 degrees Celsius in one embodiment. RF energy delivery time (RF ON time) may be selected for between 6 seconds and 18 seconds, in 2-second increments, and cooling time may be selected for between 0 seconds and 12, in 3-second increments, in one embodiment. Any other suitable ranges and combinations of ranges may be used, in alternative embodiments, and those provided here are merely examples.

For the information of the user, the impedance display 602 and stylus usage count 604 are also displayed. The back button 611 can be touched to return to the default screen 500 (FIG. 16). During treatment, the actual RF power 617 and temperature reading 609 are also shown. In alternative embodiments of the custom treatment display screen 630, one or more icons, indicators, buttons and/or windows may be moved, changed or eliminated.

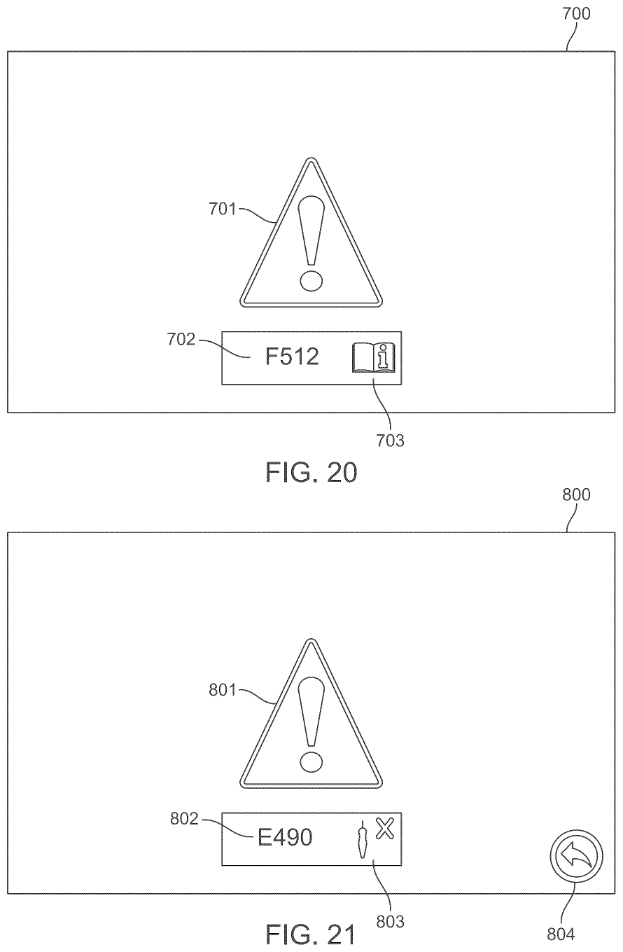

Referring now to FIG. 20, in some embodiments, the console 102 may be programmed to display a fault screen 700 when a fatal error of the system 100 occurs. The fault screen 700 may include, for example, a fault error symbol 701 that indicates a serious error has occurred, rendering the console 102 unusable. The fault screen may also include an error code 702, indicating what kind of error has occurred, and a refer to IFU (instructions for use) symbol 703. The fault error symbol 701 may be any color or combination of colors, such as a red triangle with a black exclamation point. In some embodiments, after the fault screen 700 appears, the console 102 may only be used after the user turns the console 102 off and turns it back on again.

Referring now to FIG. 21, in some embodiments, the console 102 may be programmed to display an error screen 800 when a non-fatal error of the system 100 occurs. The error screen 800 may include, for example, a caution symbol 801 that indicates an error has occurred. The error screen may also include an error code 802, indicating what kind of error has occurred, an error symbol 803, and a back button 804. The caution symbol 801 may be any color or combination of colors, such as a yellow triangle with a black exclamation point. In this embodiment, the user may touch the back button 804 to return to the previous screen being used when the error occurred, and the user may fix the error at that screen.

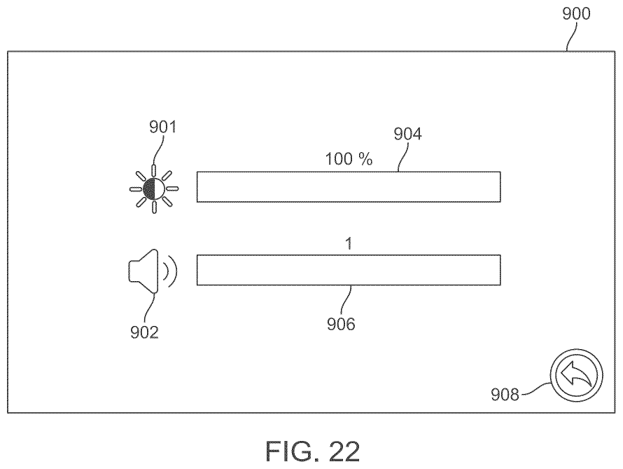

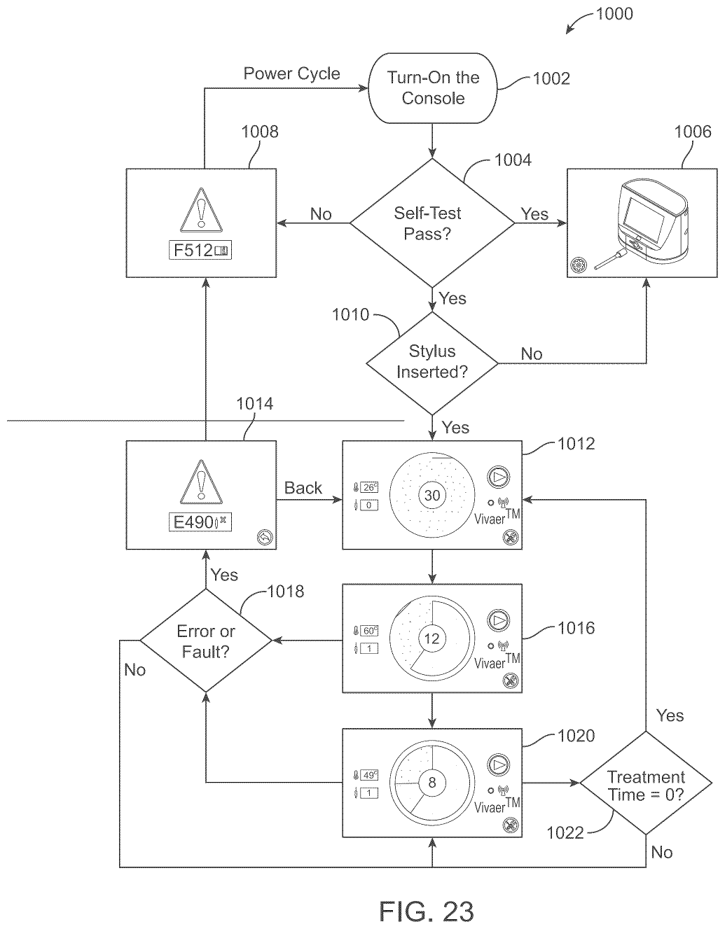

FIG. 22 is a screen shot of a settings screen 900, which may be provided on the display 108 of the console 102. In this embodiment, the settings screen 900 includes an adjust brightness icon 901, an adjust volume icon 902, a sliding bar brightness control 904 and a sliding bar volume control 906. The brightness control 904 and the volume control 906 may have any color or combination of colors. In some embodiments, a numerical indication of brightness and volume may also be included. The settings screen 900 also includes a back button 904 to allow the user to return to a previous screen.

Referring now to FIG. 22, a method 1000 of using the console 102 of the electrosurgery system 100 is illustrated. First, the user turns on 1002 the console 102. The console 102 performs a self-test 1004. If the self-test passes, the console 102 displays the standby screen 1006. If the console 102 fails the self-test, the fault screen is displayed 1008. In some embodiments, the fault screen can only be cleared by power cycling the console 102 to repeat the self-test routine. Next, assuming the self-test is passed, the user plugs the stylus 104 into the console 102. If the correct stylus is inserted 1010, the console 102 shows the default main screen 1012. The user presses the start/stop button or a foot switch, and the RF energy delivery portion of the treatment starts 1016, followed by the cooling portion 1020. When the treatment is completed 1014, the screen returns to the default main screen 1012. If an error occurs during treatment 1018, the error screen is shown 1014. The user can press the back button on the error screen to return to the default main screen 1012. If the error is fatal, the screen changes to the fault screen 1008. At various points the in method 1000, the user may also select the custom treatment screen (FIG. 19) by pressing the custom treatment button 507 on the default screen.

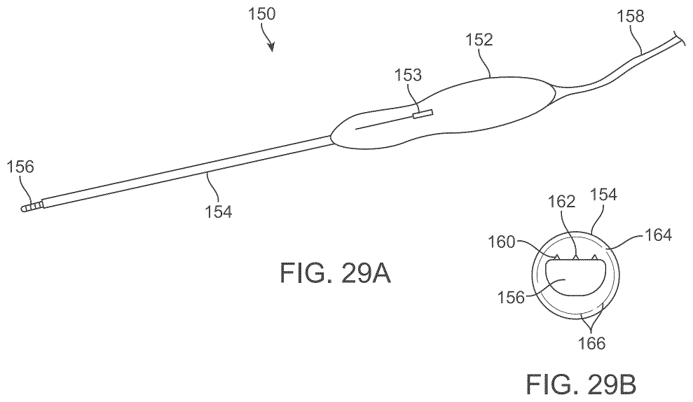

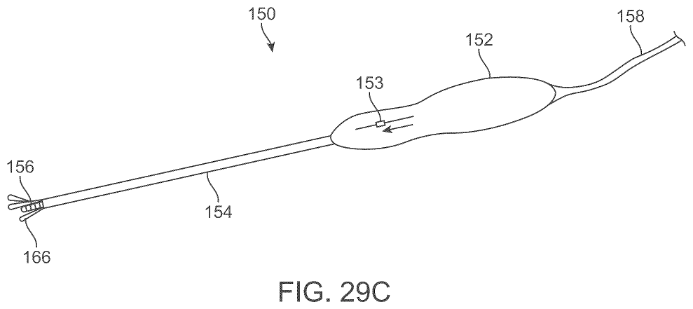

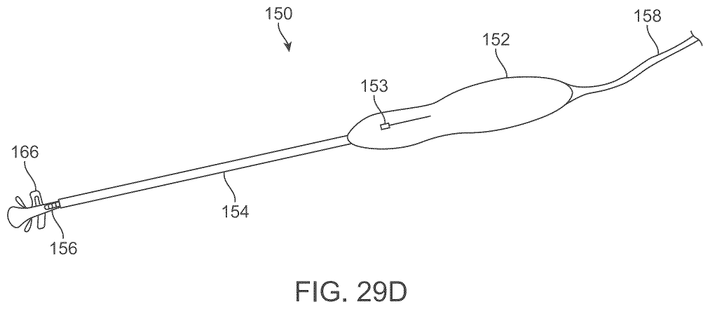

Referring now to FIGS. 29A-29D, another embodiment of a nasal airway tissue treatment stylus 150 is illustrated. As shown in FIG. 29A, the stylus 150 includes a handle 152 with a slider 153, a shaft 154, a distal tip 156 and a power cord 158. FIG. 29B is a front view of the stylus 150, showing additional features. In this embodiment, the stylus 150 includes a space 164 between the inner wall of the shaft 154 and the outer perimeter of the distal tip 156. (FIG. 29B also shows the two rows of electrodes 160 and the thermocouple 162 of the distal tip 156.) Within the space 164 and around the distal tip 156 resides an expandable wire electrode component 166, which is moveable out of the distal end of the shaft 154 to allow it to expand and be used for delivering a treatment.

In use, the stylus 150 may be used first in the configuration shown in FIG. 29A. The electrodes 160 of distal tip 156 may be used to treat tissue, such as posterior nasal nerve tissue, turbinates, nasal swell bodies and/or the like. In one embodiment, for example, the distal tip 156 may be used to treat one or more nasal turbinates in areas that are anterior to the nasal nerve that will be treated subsequently.

After treating with the stylus 150 in the FIG. 29A configuration, and referring now to FIG. 29B, the slider 153 on the handle 152 may be advanced to advance the expandable wire electrode component 166 out of the distal end of the shaft 154. FIG. 29D shows the slider 153 further advanced and the expandable wire electrode component 166 fully advanced and expanded. The expandable wire electrode component 166 may include multiple bipolar electrode pairs located at various locations along its length. Optionally, the expandable wire electrode component 166 may also include one or more thermocouples and/or multiple nerve sensors (such as electrodes), which may be used to measure a temperature of nasal mucosa and sense where nerve tissue is located under the mucosa, respectively. In some cases, all electrode pairs may be activated at the same time. Alternatively, only one or more selected electrode pairs may be activated. In some cases, the expandable wire electrode component 166 may be used to determine the location of nerves and then only bipolar electrode pairs located directly over those nerves may be activated.

In one example, after the stylus 150 has been used in the initial configuration shown in FIG. 29A to treat one or more turbinates, swell bodies or the like, the stylus 150 may be changed to the expanded configuration shown in FIG. 29D. In that configuration, the expandable wire electrode component 166 may be used to treat posterior nasal nerves and/or any other nerve tissue the physician wants to treat. In other words, the stylus 150 is used to treat nerve tissue in the FIG. 29D configuration and used to treat any other tissues in the FIG. 29A configuration. This is only one example, however, and a physician may use the stylus 150 in any suitable manner. After treatment with the expandable wire electrode component 166 is complete, the expandable wire electrode component 166 may be pulled back into the shaft by sliding the slider 153 proximally. It may also be possible to treat further in the FIG. 29A configuration after treatment with, and retraction of, the expandable wire electrode component 166.

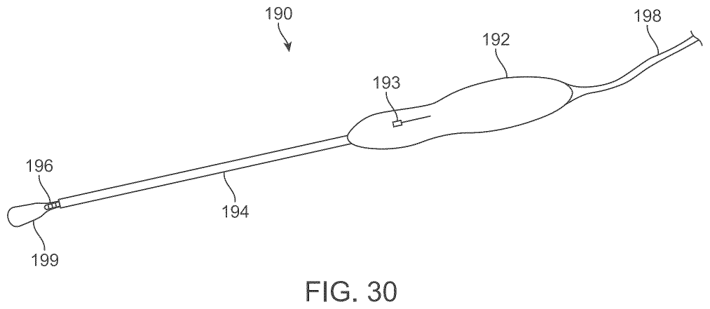

Referring now to FIG. 30, an alternative embodiment of a nasal airway treatment stylus 190 is similar to the one just described, except that rather than the expandable wire electrode component 166, this embodiment of the stylus 190 includes an expandable cryotherapy balloon 199. The stylus 190 also includes a handle 192 with a slider 193, a shaft 194, a distal tip 196 and a power cord 198. In this embodiment, the cryotherapy balloon 199 can be advanced out of the distal end of the shaft 194 using the slider 193. A source of cryogenic substance, such as nitrous oxide in a small canister, may be attached to the handle 192 and transmitted through a lumen in the shaft 194 to inflate the cryotherapy balloon 199. The inflated cryotherapy balloon 199 will absorb energy from the nasal airway tissues and thus can be used to ablate posterior nasal nerves and/or any other target nerve tissue or other target tissues. When a treatment is complete, the cryogenic substance may be evacuated from the cryotherapy balloon 199, causing it to deflate, and the stylus 190 may be removed from the nose. Optionally, the deflated cryotherapy balloon 199 may be pulled back into the shaft 194 before removal of the stylus 190, but that may not be necessary. As with the previous embodiment, any suitable nasal airway tissue may be treated with the distal tip 196 of the stylus 190 before inflating the cryotherapy balloon 199 for additional tissue treatment.

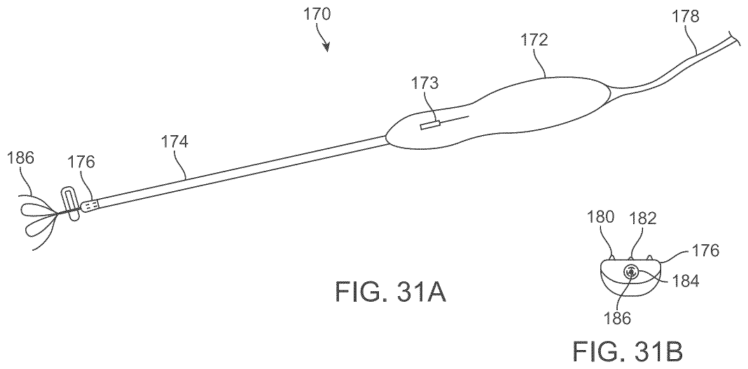

With reference now to FIGS. 31A and 31B, another alternative embodiment of a nasal airway tissue treatment device 170 is illustrated in perspective view (FIG. 31A) and front view (FIG. 31B). In this embodiment, the stylus 170 includes a handle 172 with a slider 173, a shaft 174, a distal tip 176, an expandable wire electrode component 186, and a power cord 178. As best seen in FIG. 31B, the distal tip 176 includes two rows of bipolar RF electrode pairs 180, a thermocouple 182 and an opening 184 which leads directly into a lumen in the shaft 174. The expandable wire electrode component 186 is located inside the opening 184 and the lumen (not visible on these drawings) and may be advanced out of the opening 184 with slider 173 to allow the expandable wire electrode component 186 to expand and be used for treatment. All of the same features and methods of use described above may be applied to this embodiment of the stylus 170, the primary difference being that the expandable wire electrode component 186 is located within the distal tip 176 and advances out of the opening 184. The expandable wire electrode component 186 may have any suitable size, shape and configuration of wires, according to various embodiments.

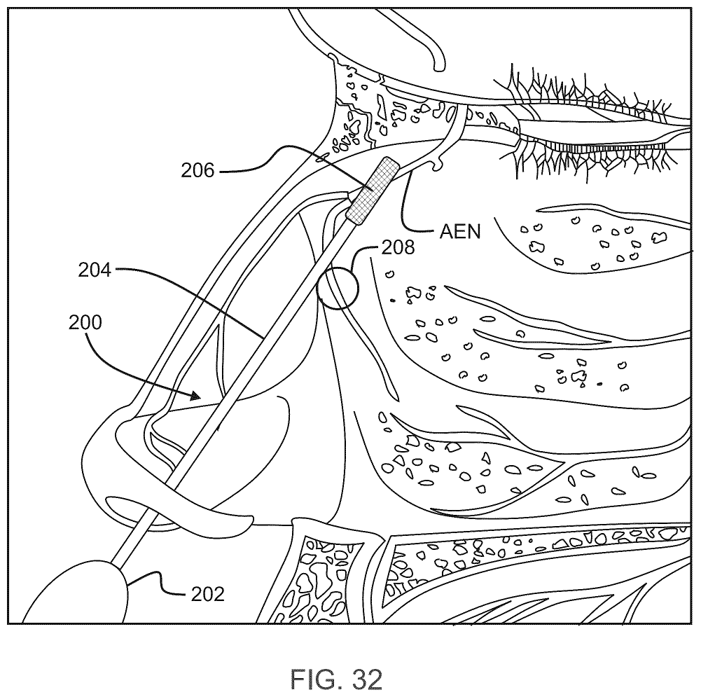

FIG. 32 is a sagittal cross-section of a nasal cavity, illustrating the anterior ethmoidal nerve AEN and a treatment device 200 in position to deliver energy to, and thus treat, the anterior ethmoidal nerve AEN. The treatment device 200 includes a handle 202 (only part of which is shown), a shaft 204, and a treatment tip 206. On one side of the treatment tip 206, facing the anterior ethmoidal nerve AEN so not visible in the drawing, are one or more energy delivery members. For example, the energy delivery members may be two or more RF electrodes, which act as bipolar electrodes. Some embodiments include multiple pairs of RF electrodes.

In various examples, the RF energy can be delivered in different types of modalities, such as pulsed, sinusoidal, etc. In other embodiments, the treatment tip 206 may include a monopolar RF probe, cryotherapy device (e.g., a small cryotherapy balloon), DC current delivery, heat delivery, chemical delivery (e.g., Botox), an ultrasound transducer, or any other suitable energy delivery member(s).

A method of using the treatment device 200 may involve advancing the treatment tip 206 through a nostril, into the nasal cavity, and to a desired treatment position, in contact with nasal mucosa overlying the anterior ethmoidal nerve AEN. Radiofrequency energy (or other forms of energy in alternative embodiments) may then be delivered for anywhere from a few seconds to several minutes, to ablate the anterior ethmoidal nerve AEN or to otherwise affect it without ablating. After a first treatment, the physician may relocate the treatment tip 206 to another portion of the anterior ethmoidal nerve AEN, a branch of the anterior ethmoidal nerve AEN, a posterior nasal nerve, or any other nerve or structure in the nasal cavity, and deliver energy at that second location. This process may be repeated as many times as the physician desires. For example, it may be advantageous to use the treatment device 200 to ablate the anterior ethmoidal nerve AEN in multiple locations, to ablate one or more branches of the anterior ethmoidal nerve AEN, and to at least partially ablate the posterior nasal nerve PNN, all in the same treatment. In alternative embodiments, a treatment might be directed only at the anterior ethmoidal nerve AEN and its branches, since they are typically easier to access than the posterior nasal nerve. Any combination of treatment numbers and nerves and/or other tissues treated may be performed, according to various embodiments of the treatment method. For instance, treatment can be directed to an area 208 of the nerve in some embodiments. Once a treatment is complete in one nostril, the treatment device 200 may be removed, and the other nostril may be treated.

The example treatment device 200 can be used to treat the anterior ethmoidal nerve AEN in conjunction with various disease states in a unified airway approach. Examples include, without limitation: asthma; chronic rhino sinusitis (CRS); chronic cough; sneezing; eustachian tube dysfunction; emphysema; and chronic obstructive pulmonary disease (COPD). Many other uses are possible.

Although this application is believed to be complete and accurate, any suitable changes may be made to any of the described embodiments and features described above, without departing from the scope of the invention.

What is claimed is:

1. A method for treating a nasal airway to ameliorate one or more symptoms of rhinitis in a patient, the method comprising:
advancing a treatment tip of a treatment device into a nostril of the patient;
contacting nasal mucosa with the treatment tip at a first location overlying an anterior ethmoidal nerve;
delivering energy from the treatment tip, to change a property of the anterior ethmoidal nerve; and
removing the treatment tip from the nostril.

2. The method of claim 1, further comprising:
moving the treatment tip to a second location overlying additional nerve tissue selected from the group consisting of the anterior ethmoidal nerve, a branch of the anterior ethmoidal nerve, and a posterior nasal nerve; and
delivering energy from the treatment tip at the second location.

3. The method of claim 2, further comprising:
moving the treatment tip to a third location; and
delivering energy from the treatment tip at the third location, wherein the energy is delivered in a pulsed or sinusoidal modality.

4. The method of claim 3, wherein the third location is selected from the group consisting of a turbinate, a swell body, and a nasal septum.

5. The method of claim 1, wherein changing the property of the anterior ethmoidal nerve comprises ablating the anterior ethmoidal nerve.

6. The method of claim 1, wherein delivering the energy comprises delivering an energy type selected from the group consisting of radiofrequency, DC current, microwave, heat, cryotherapy, ultrasound, and chemical energy.

7. The method of claim 1, further comprising sensing at least one of temperature or impedance with a sensor of the treatment device.

8. The method of claim 7, further comprising automatically shutting off delivery of energy from the treatment device if the temperature or the impedance exceeds a preset maximum.

9. The method of claim 1, further comprising bending a shaft of the treatment device before advancing the treatment tip into the nostril.

10. The method of claim 1, further comprising treating the nasal airway in conjunction with treatment of one or more of: asthma; chronic rhino sinusitis (CRS); chronic cough; sneezing; eustachian tube dysfunction; emphysema; and chronic obstructive pulmonary disease (COPD).

11. A device for treating an anterior ethmoidal nerve to ameliorate one or more symptoms of rhinitis, the device comprising:
a handle;
a shaft extending from the handle; and
an energy delivery treatment tip extending from a distal end of the shaft.

12. The device of claim 11, wherein the shaft is malleable and has a width of 4 millimeters to 5 millimeters.

13. The device of claim 11, wherein the energy delivery treatment tip comprises at least two radiofrequency electrodes, and wherein the device further comprises a power cord connected at one end to the handle and including a connector at an opposite end for connecting to an outlet of a radiofrequency console.

14. The device of claim 11, wherein the energy delivery treatment tip is configured to deliver energy selected from the group consisting of radiofrequency, DC current, heat, microwave, ultrasound, cryotherapy, and chemical energy.

15. The device of claim 11, wherein the energy delivery treatment tip comprises an expandable cryotherapy balloon.

16. The device of claim 11, wherein a length of the shaft is selected to extend from a first end that is attached to the handle, located just outside a nostril of a patient, to a second end, attached to the energy delivery treatment tip located at the anterior ethmoidal nerve in a nasal cavity of the patient.

17. The device of claim 11, wherein treatment of the anterior ethmoidal nerve is performed in conjunction with treatment of one or more of: asthma; chronic rhino sinusitis (CRS); chronic cough; sneezing; eustachian tube dysfunction; emphysema; and chronic obstructive pulmonary disease (COPD).

18. The device of claim 11, wherein the energy delivery treatment tip is configured to deliver energy in a pulsed or sinusoidal modality.
